(19) United States
(12) Patent Application Publication
(10) Pub. No.: US 2015/0105636 A1
(43) Pub. Date: Apr. 16, 2015

(54) SYSTEM AND METHOD FOR EMERGENCY RESUSCITATION

(71) Applicant: Covidien LP, Mansfield, MA (US)

(72) Inventors: Sarah Hayman, Boulder, CO (US); Paulo E.X. Silveira, Boulder, CO (US); Jill Klomhaus Anderson, Longmont, CO (US); Yu-Jung Pinto, Boulder, CO (US)

(73) Assignee: Covidien LP, Mansfield, MA (US)

(21) Appl. No.: 14/051,022

(22) Filed: Oct. 10, 2013

(51) Int. Cl. A61B 5/1455; A61B 5/00; A61B 5/0205

(52) U.S. Cl. CPC A61B 5/14552; A61B 5/0205; A61B 5/7415; A61B 5/7475; A61B 5/742; A61B 5/02427
USPC 600/324; 600/323

(57) ABSTRACT

According to various embodiments, a regional oximetry sensor may include a light emitting element configured to emit light, a light detector configured to receive the light and generate a signal based on the received light. The regional oximetry sensor, itself or in conjunction with a monitor, may enable communicating adjustments in the administration of CPR to a patient based on one or more characteristics (e.g., pulse amplitude or pulse rate) of the signal generated by the regional oximetry sensor.

### BACKGROUND

The present disclosure relates generally to emergency resuscitation and, more particularly, to sensors and/or monitors and/or algorithms configured to assist a person in performing emergency resuscitation.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In many medical emergencies, a person's heart may stop pumping on its own. The person may need emergency resuscitation such as cardiopulmonary resuscitation (CPR) to sustain the life of the person by manually maintaining intact brain function. Typically, CPR involves manually pumping the chest (i.e., chest compressions) to force blood through the cardiovascular system to organs such as the brain. CPR also involves occasionally blowing oxygenated air (i.e., administered breaths or artificial respiration) into the lungs of the person so that oxygen may be absorbed into the bloodstream. However, the person administering the CPR, whether a trained emergency responder or a person with little training or experience in administering CPR, has little to no feedback as to the effectiveness of the CPR (e.g., quality of chest compressions or applied breaths) being administered. Consequently, the CPR may not be administered as effectively as possible.

### BRIEF DESCRIPTION OF THE DRAWINGS

Advantages of the disclosed techniques may become apparent upon reading the following detailed description and upon reference to the drawings in which:

FIG. 1 is a front view of an embodiment of a monitoring system configured to be used with a sensor for regional saturation, in accordance with an aspect of the present disclosure;

FIG. 2 is a block diagram of the monitoring system of FIG. 1 (e.g., sensor coupled to monitor via wired connection), in accordance with an aspect of the present disclosure;

FIG. 3 is a graphical representation of a signal received from the sensor of FIG. 1;

FIG. 4 is a graphical representation of compression force of administered chest compressions versus pulse amplitude of pulses of the signal received from the sensor;

FIG. 5 is a block diagram of the monitoring system of FIG. 1 (e.g., sensor wirelessly coupled to monitor), in accordance with an aspect of the present disclosure;

FIG. 6 is a process flow diagram of an embodiment of a method for using the monitoring system of FIG. 1;

FIG. 7 is a process flow diagram of an embodiment of a method for determining the effectiveness of CPR administered to a patient using the monitoring system of FIG. 1; and

FIG. 8 is a front view of an embodiment of a regional saturation monitor, in accordance with an aspect of the present disclosure.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Also, as used herein, the term “over” or “above” refers to a component location on a sensor that is closer to patient tissue when the sensor is applied to the patient.

The present embodiments relate to emergency response kits (i.e., emergency response components described below provided or sold as a single unit for use in an emergency response) that may include a sensor and/or monitor to monitor one or more physiological characteristics (e.g., regional oxygen saturation (rSO₂)) of a patient (i.e., person receiving emergency resuscitation such as CPR). The sensors described herein may incorporate one or more emitters and one or more detectors for determining the level of blood oxygen saturation in a particular region, such as a cerebral or somatic region, which may be referred to as regional oximetry. In addition, characteristics or features of the signal acquired by the sensor from the patient may provide useful feedback related to the administration of the CPR. For example, these characteristics or features may include a pulse rate (e.g., frequency) and pulse amplitude of the signal that relate to the quality of administered chest compressions (e.g., appropriate location of chest compressions and/or strength of chest compressions), respectively. In addition, an rSO₂ value derived from the signal may be used to provide useful feedback related to the administration of the CPR. For example, the rSO₂ value may be related to the quality (e.g., effectiveness with regards to volume or frequency) of the breaths (i.e., artificial respiration) administered during the CPR. The sensor and/or monitor may compare a particular signal characteristic to a range (e.g., optimal range) or the rSO₂ value to a threshold to determine whether a component of the CPR needs to be altered (e.g., chest compressions and/or breaths). The optimal ranges may be based on characteristics of the patient (e.g., infant vs. adult, size of patient, age, etc.).

The feedback with regards to the administration of the CPR may be communicated from the sensor and/or monitor (e.g., via a speaker and/or a display). In certain embodiments, the sensor may communicate via a wired connection (e.g., cable) or wirelessly with the monitor. Alternatively, the sensor may include some or all of the hardware (e.g., speaker, display, memory, processing device, etc.) and/or software to analyze the characteristics of the signals and to communicate any feedback (e.g., adjustments) to the person administering the CPR. It should be noted that CPR as described herein includes the components of administering chest compressions and artificial respiration. However, the techniques and systems described herein may also be utilized in conjunction with any type of CPR (e.g., CPR administered without artificial respiration). In addition, additional emergency response techniques may be utilized with CPR (e.g., defibrillation).

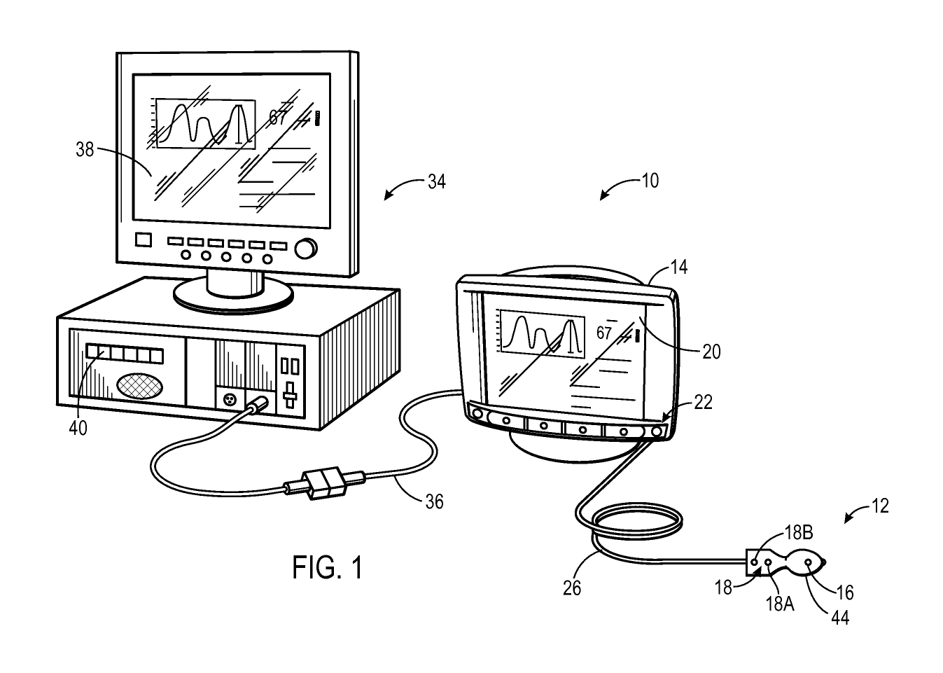

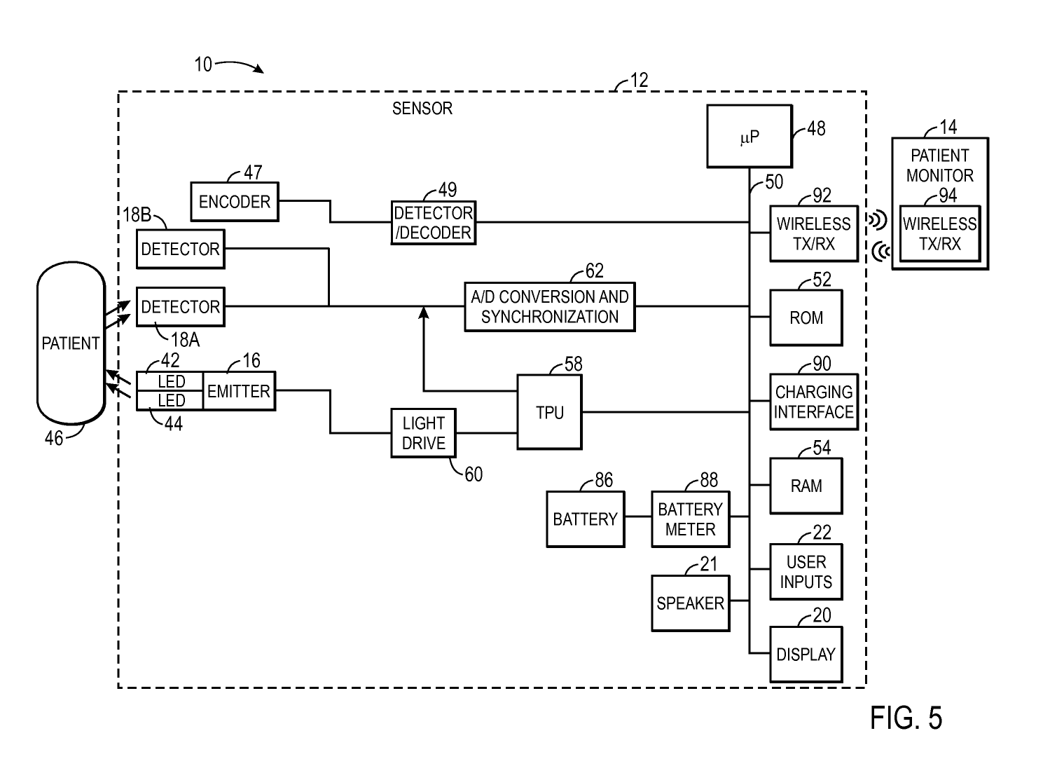

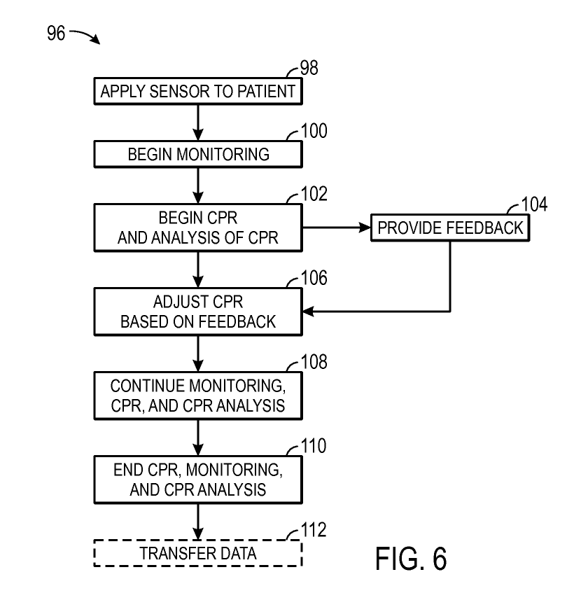

By way of example, an INVOS® cerebral/somatic sensor, such as an OxyAlert™ NIR sensor by Somanetics Corporation or a SomaSensor® by Somanetics Corporation, which may include one or more emitters and a pair of detectors for determining site-specific oxygen levels, may represent sensors used in the described techniques and systems. Example systems incorporating a sensor and/or monitor capable of performing regional oximetry and communicating real-time feedback (i.e., as the CPR is performed) related to the administration of CPR are discussed with respect to FIGS. 1, 2, and 4. Example methods for using these systems and sensors are discussed with respect to FIGS. 5 and 6.

With this in mind, FIG. 1 depicts an embodiment of a patient monitoring system 10 that may be used in conjunction with a medical sensor 12. Although the depicted embodiments relate to sensors for use on a patient's head, it should be understood that, in certain embodiments, the features of the sensor 12 as provided herein may be incorporated into sensors for use on other tissue locations, such as the back, the stomach, the heel, the ear, an arm, a leg, or any other appropriate measurement site. In addition, although the embodiment of the patient monitoring system 10 illustrated in FIG. 1 relates to photoplethysmography or regional oximetry, the system 10 may be configured to obtain a variety of medical measurements with a suitable medical sensor. For example, the system 10 may additionally be configured to determine patient electroencephalography (e.g., a bispectral index), or any other desired physiological parameter such as water fraction or hematocrit.

As noted, the system 10 includes the sensor 12 that is communicatively coupled to a patient monitor 14. The sensor 12 may be reusable, entirely disposable, or include disposable portions. If the sensor 12 is reusable, it may include a disposable adhesive pad that may be replaced. Although only one sensor 12 is shown coupled to the monitor 14 in FIG. 1, in other embodiments, two, three, four, or more sensors 12 may be coupled to the monitor 14. For example, two sensors 12 may be used for cerebral oximetry and simultaneously two other sensors 12 used for somatic oximetry. As shown in FIG. 1, the sensor 12 includes an emitter 16 and a pair of detectors 18. The emitter 16 and detectors 18 of the sensor 12 are coupled to the monitor 14 via a cable 26 coupled to the monitor 14. The cable 26 may interface directly with the sensor 12 and may include a plurality of conductors (e.g., wires). In certain embodiments, the sensor 12 may be configured to store patient-related data, such as historical regional oximetry data (e.g., rSO₂ values) and signal characteristics related to the administration of CPR.

The monitor 14 includes a monitor display 20 configured to display information regarding the physiological parameters monitored by the sensor 12, information about the system, and/or alarm indications. In addition, the monitor display 20 may be configured to communicate information related to the CPR being administered to the patient. For example, information related to chest compressions (e.g., “change location of compressions”, “compression too light”, “compression too hard”, “slow down compressions”, “speed up compressions”, etc.) and/or artificial respiration may be displayed on the display 20. This information may relate to changing a location of the chest compressions, the amount of force applied during the chest compressions (e.g., too light, too hard, etc.), and/or the effectiveness of the administered breaths (e.g., an amount, frequency, etc.). The information may be displayed via text, images, and/or color-coded indicators. The monitor 14 may also include a speaker 21 to communicate information related to the CPR being administered to the patient. For example, the speaker 21 may communicate audible instructions (e.g., “change location of compressions”, “compression too light”, “compression too hard”, “slow down compressions”, “speed up compressions”, etc.). In addition, the speaker 21 may emit a sound (e.g., beep) to reflect a detected pulse. In some embodiments, a pitch, tone, or other characteristic of the sound may be varied to indicate chest compressions are being administered too fast, too slow, or at a correct rate. The monitor 14 may include various input components 22, such as knobs, switches, keys and keypads, buttons, touchscreen, etc., to provide for operation and configuration of the monitor 14. The input components 22 may enable the inputting and/or adjusting of patient characteristics (e.g., patient age, size, condition, etc.), inputting that the sensor 12 has been applied to the patient, inputting a beginning and/or end of the administration of CPR to the patient, and/or inputting and/or adjusting ranges, values, and/or thresholds related to determining the effectiveness of the administered CPR (e.g., optimal pulse frequency range, optimal pulse rate range, optimal rSO₂ value, threshold, or range). In certain embodiments, the sensor 12 may include input components for inputting/and or adjusting this information. The monitor 14 also includes a processor that may be used to execute code, such as code for implementing various monitoring functionalities enabled by the sensor 12. As discussed below, for example, the monitor 14 may be configured to process signals generated by the detectors 18 to estimate the amount of oxygenated vs. de-oxygenated hemoglobin in a monitored region of the patient (e.g., brain). In addition, the monitor 14 may be configured to relate the rSO₂ value to a component of the CPR (e.g., artificial respiration). Further, the monitor 14 may be configured to analyze the signals generated by the detectors 18 to relate signal characteristics (e.g., pulse rate and/or pulse frequency) to one or more components of the CPR (e.g., chest compressions). In some embodiments, the sensor 12 may include a processor that may be used to execute code stored in a memory of the sensor 12 to perform all or some of the functionalities described throughout related to calculating an rSO₂ value and/or analyzing signals characteristics related to one or more components of the CPR.

The monitor 14 may be any suitable monitor, such as an INVOS® System monitor available from Somanetics Corporation. Furthermore, to upgrade conventional operation provided by the monitor 14 to provide additional functions, the monitor 14 may be coupled to a multi-parameter patient monitor 34 via a cable 36 connected to a sensor input port. In addition to the monitor 14, or alternatively, the multi-parameter patient monitor 34 may be configured to calculate physiological parameters and to provide a central display 38 for the visualization of information from the monitor 14 and from other medical monitoring devices or systems. The multi-parameter monitor 34 includes a processor that may be configured to execute code. The multi-parameter monitor 34 may also include various input components 40, such as knobs, switches, keys and keypads, buttons, touchscreen, etc., to provide for operation and configuration of the a multi-parameter monitor 34. In addition, the monitor 14 and/or the multi-parameter monitor 34 may be connected to a network to enable the sharing of information with servers or other workstations (e.g., electronic medical records).

In certain embodiments, the sensor 12 may be a wireless sensor 12. Accordingly, the wireless sensor 12 may establish a wireless communication with the patient monitor 14, the multi-parameter patient monitor 34, and/or network using any suitable wireless standard. By way of example, the wireless module may be capable of communicating using one or more of the ZigBee standard, WirelessHART standard, Bluetooth standard, IEEE 802.11x standards, or MiWi standard.

As provided herein, the sensor 12 may be configured to perform regional oximetry. Indeed, in one embodiment, the sensor 12 may be an INVOS® cerebral/somatic sensor available from Somanetics Corporation. In regional oximetry, by comparing the relative intensities of light received at two or more detectors, it is possible to estimate the blood oxygen saturation of hemoglobin in a region of a body. For example, a regional oximeter may include a sensor to be placed on a patient's forehead and may be used to calculate the oxygen saturation of a patient's blood within the venous, arterial, and capillary systems of a region underlying the patient's forehead (e.g., in the cerebral cortex). As illustrated in FIGS. 1-3, the sensor 12 may include the emitter 16 and the two detectors 18: one detector 18A that is relatively “close” to the emitter 16 and another detector 18B that is relatively “far” from the emitter 16. Light intensity of one or more wavelengths may be received at both the “close” and the “far” detectors 18A and 18B. Thus, the detector 18A may receive a first portion of light and the detector 18B may receive a second portion of light. Each of the detectors 18 may generate signals indicative of their respective portions of light. For example, the resulting signals may be contrasted to arrive at a regional saturation value that pertains to additional tissue through which the light received at the “far” detector 18B passed (tissue in addition to the tissue through which the light received by the “close” detector 18A passed, e.g., the brain tissue) when it was transmitted through a region of a patient (e.g., a patient's cranium). Surface data from the skin and skull is subtracted out to produce a regional oxygen saturation (rSO₂) value for deeper tissues. In certain embodiments, the sensor 12 and/or monitor 14 may be configured to select the desired (e.g., strongest) signals from the signals provided by the detectors 18 for use in determining the rSO₂ value and/or analyzing the signal characteristics related to one or more components of the CPR being administered.

In certain embodiments, sensor 12 may be entirely or partially reusable and integrated with monitor 14 in a single unit possessing its own display and requiring no cable 26. The integrated monitor would be a standalone unit, strapped to the head of the patient and in direct view of the operator. Such embodiment would present the advantages of greater mobility and reduced number of parts.

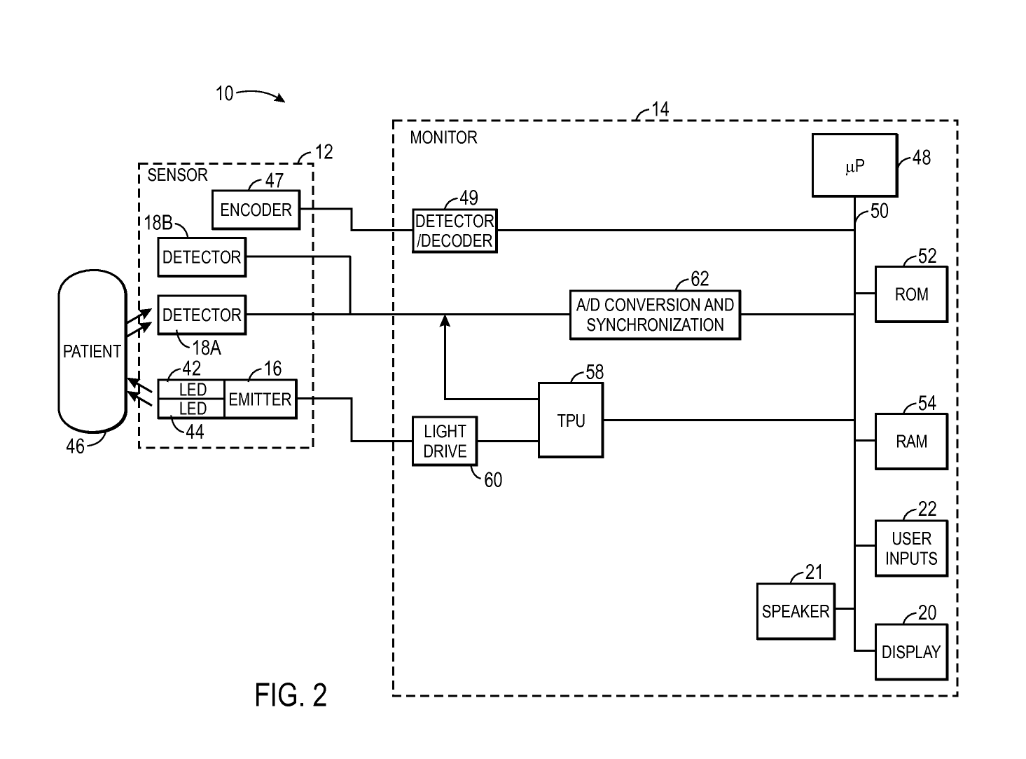

Turning to FIG. 2, a simplified block diagram of the medical system 10 is illustrated in accordance with an embodiment. The sensor 12 may include optical components in the forms of the emitter 16 and detectors 18. The emitter 16 and the detectors 18 may be arranged in a reflectance or transmission-type configuration with respect to one another. However, in embodiments in which the sensor 12 is configured for use on a patient's forehead, the emitter 16 and detectors 18 may be in a reflectance configuration. An emitter 16 may also be a light emitting diode, superluminescent light emitting diode, a laser diode, or a vertical cavity surface emitting laser (VCSEL). An emitter 16 and the detectors 18 may also include optical fiber sensing elements. Also, the emitter 16 may include two light emitting diodes (LEDs) 42 and 44 that are capable of emitting at least two wavelengths of light, e.g., red or near infrared light. In one embodiment, the LEDs 42 and 44 emit light in the range of 600 nm to about 1000 nm. In a particular embodiment, the one LED 42 is capable of emitting light at 730 nm and the other LED 44 is capable of emitting light at 810 nm. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In any suitable configuration of the sensor 12, the detectors 18A and 18B may be an array of detector elements that may be capable of detecting light at various intensities and wavelengths. In one embodiment, light enters the detector 18 (e.g., detector 18A or 18B) after passing through the tissue of the patient 46. In another embodiment, light emitted from the emitter 16 may be reflected by elements in the patient's tissue to enter the detector 18. The detector 18 may convert the received light at a given intensity, which may be directly related to the absorbance and/or reflectance of light in the tissue of the patient 46, into an electrical signal. That is, when more light at a certain wavelength is absorbed, less light of that wavelength is typically received from the tissue by the detector 18, and when more light at a certain wavelength is reflected, more light of that wavelength is typically received from the tissue by the detector 18. After converting the received light to an electrical signal, the detector 18 may send the signal to the monitor 14, where physiological characteristics may be calculated based at least in part on the absorption and/or reflection of light by the tissue of the patient 46.

In certain embodiments, the medical sensor 12 may also include an encoder 47 that may provide signals indicative of the wavelength of one or more light sources of the emitter 16, which may allow for selection of appropriate calibration coefficients for calculating a physical parameter such as blood oxygen saturation. The encoder 47 may, for instance, include a coded resistor, an electrically erasable programmable read only memory (EEPROM), or other coding device (such as a capacitor, inductor, programmable read only memory (PROM), RFID, parallel resident currents, or a colorimetric indicator) that may provide a signal to a microprocessor 48 related to the characteristics of the medical sensor 12 to enable the microprocessor 48 to determine the appropriate calibration characteristics of the medical sensor 12. Further, the encoder 47 may include encryption coding that prevents a disposable part of the medical sensor 12 from being recognized by a microprocessor 48 unable to decode the encryption. For example, a detector/decoder 49 may translate information from the encoder 47 before the processor 48 can properly handle it. In some embodiments, the encoder 47 and/or the detector/decoder 48 may not be present.

In certain embodiments, the sensor 12 may include circuitry that stores patient-related data (e.g., rSO₂) and provides the data when requested. The circuitry may be included in the encoder 47 or in separate memory circuitry within the sensor 12. Examples of memory circuitry include, but are not limited to, a random access memory (RAM), a FLASH memory, a PROM, an EEPROM, a similar programmable and/or erasable memory, any kind of erasable memory, a write once memory, or other memory technologies capable of write operations. In one embodiment, patient-related data, such as the rSO₂ values, trending data, or patient monitoring parameters, may be actively stored in the encoder 47 or memory circuitry.

Returning to FIG. 2, signals from the detector 18 and/or the encoder 47 may be transmitted to the monitor 14. By way of example, the monitor 14 shown in FIG. 2 may be an INVOS® System monitor 14 available from Somanetics Corporation. The monitor 14 may include one or more processors 48 coupled to an internal bus 50. Also connected to the bus 50 may be a ROM memory 52, a RAM memory 54, and the display 20. A time processing unit (TPU) 58 may provide timing control signals to light drive circuitry 60, which controls when the emitter 16 is activated, and if multiple light sources are used, the multiplexed timing for the different light sources. The received signal from the detector 18 may be passed through analog-to-digital conversion and synchronization 62 under the control of timing control signals from the TPU 58. Specifically, the signal may undergo synchronized demodulation and optionally amplification and/or filtering. For example, the LEDs 42 and 44 may be driven out-of-phase, sequentially and alternatingly with one another (i.e., only one of the LEDs 42 and 44 being driven during the same time interval) such that the detector 18 receives only resultant light spectra emanating from one LED at a time. Demodulation 62 of the signal enables the data associated with the LEDs 42 and 44 to be distinguished from one another. After demodulation, the digital data may be downloaded to the RAM memory 54.

In some embodiments, the processor 48 may determine the placement of the sensor 12 on the patient 46 by detecting activity using various algorithms (e.g., “sensor off” algorithms, pulse detection algorithms, etc.). Alternatively, the processor 48 may be configured to receive user input via the input components 22 that indicate the placement of the sensor 12 on the patient 46. In addition, the processor 48 may be configured to receive user input via the input components 22 that indicate the beginning and/or end of the administration of the CPR to the patient 46.

In an embodiment, based at least in part upon the received signals corresponding to the light received by detector 18, the processor 48 may calculate the oxygen saturation (e.g., regional oxygen saturation) using various algorithms. These algorithms may use coefficients, which may be empirically determined. For example, algorithms relating to the distance between an emitter 16 and various detector elements in a detector 18 may be stored in the ROM memory 52 and accessed and operated according to processor 48 instructions.

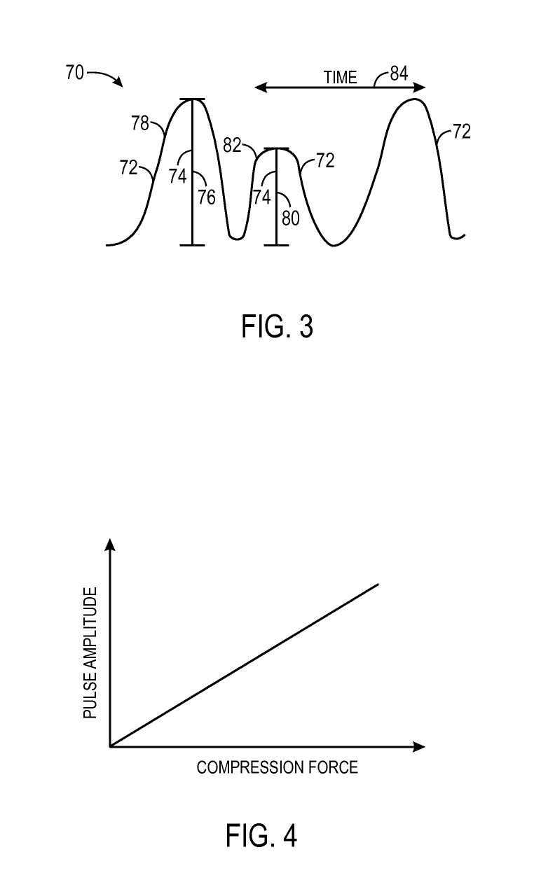

In addition, the processor 48 may select the strongest signal received from the detectors 18 (e.g., from a shallow detector or a deep detector) for calculating the oxygen saturation and further analysis of the signal. For example, the processor 48 may analyze characteristics of the signal and relate them to components of the CPR presently being administered to the patient 46 using various algorithms. Specifically, the processor 48 may calculate the pulse amplitude and/or pulse rate of the signal and relate these to the chest compressions administered to the patient 46. FIG. 3 provides an example of a signal 70 obtained from the sensor 12. The signal 70 includes multiple pulses 72. Each pulse 72 includes a pulse amplitude 74 (i.e., peak to peak amplitude for the pulse 72). The pulse amplitude 74 may be linearly related to the force applied during the chest compression (see FIG. 4) and/or the effectiveness of the chest compression (e.g., location of the chest compression). For example, a stronger chest compression may result in a larger pulse amplitude 74 (see pulse amplitude 76 for pulse 78 in FIG. 3) relative to the pulse amplitude 74 from a weaker chest compression (see pulse amplitude 80 for pulse 82 in FIG. 3). Similarly, a chest compression administered in the wrong location (e.g., off-center) may result in a smaller pulse amplitude 74 (see pulse amplitude 80 for pulse 82 in FIG. 3) than a pulse amplitude 74 from a chest compression administered in the proper location (see pulse amplitude 76 for pulse 78 in FIG. 3). FIG. 3 also illustrates the pulse rate (i.e., frequency) of the signal 70 (i.e., the number of pulses 72 within a defined period of time 84). Generally the frequency of the detected pulse (i.e., signal 70) may be the same as the frequency of the chest compressions administered during the CPR. Returning to FIG. 2, the processor 48 may be configured to relate the rSO₂ value to a component of the CPR (e.g., artificial respiration).

Further, the processor 48 may be configured to compare the rSO₂ value and characteristics of the signal to threshold values and/or ranges and communicate information related to the administered CPR (e.g., via the speaker 21 and/or display 20 as described above) based on these comparisons. For example, the processor 48, via the speaker 21 and/or display 20, may communicate to the person administering the CPR to adjust one or more components of the CPR (e.g., chest compressions and/or artificial respiration). The processor 48 may compare the rSO₂ value to a threshold value to determine if the rSO₂ value is lower than the threshold value and communicate to the person administering the CPR to adjust (e.g., increase or decrease) the frequency and/or intensity of breaths administered during the CPR if the rSO₂ value is lower than the threshold value. An rSO₂ value below the threshold value may be indicative that the brain of the patient 46 is not receiving enough oxygen. Alternatively, the processor 48 may be configured to determine if the rSO₂ value falls within an optimal range. The processor 48 may also keep track of the number of compressions administered and communicates when artificial respiration should be administered. The processor 48 may also compare the pulse amplitude of the signal to a pulse amplitude range (e.g., optimal pulse amplitude range) and communicate (e.g., via the speaker 21 and/or display 20 as described above) to the person administering the CPR to adjust chest compressions administered during the CPR if the pulse amplitude is not within the pulse amplitude range. The pulse amplitude range may be based on a nominal value determined through empirical data or inputted by a user. Additionally, the processor 48 may be configured to compare the pulse rate of the signal to a pulse rate range (e.g., optimal pulse rate range) and to communicate (e.g., via the speaker 21 and/or display 20 as described above) to the person administering the CPR to adjust chest compressions administered during the CPR if the pulse rate is not within the pulse rate range. The pulse rate range may be based on a recommended pulse rate (e.g., 100 compressions per minute). The pulse amplitude range and pulse rate range may be stored within the ROM memory 52. In addition, the user may adjust and/or enter a desired pulse amplitude range and pulse rate range. Alternatively, the user may input patient characteristics (e.g., age, size, etc.) and the processor 48 may be configured to adjust the pulse amplitude range and the pulse rate range based on these patient characteristics. Alternatively, the processor 48 may detect the type of sensor that is in use (e.g. adult, pediatric, infant) and adjust the target amplitudes, rates and ranges based on the patient population for the selected sensor.

Furthermore, one or more functions of the monitor 14 may also be implemented directly in the sensor 12 as illustrated in FIG. 5. The sensor 12 in FIG. 5 includes the components of the monitor 14 shown in FIG. 2. However, the sensor 12, in certain embodiments, may include only some of these components. For example, in some embodiments, the sensor 12 may includes the processor 48 capable of calculating the physiological characteristics (e.g., rSO₂ value) from the signals obtained from the patient 46. In addition, the processor 48 is capable of analyzing characteristics of the signals with regard to one or more components of CPR being administered to the patient 46 and communicating information (e.g., via display 20 and/or speaker 21) related to the CPR as described above. A battery 86 may supply the sensor 12 with operating power. By way of example, the battery 86 may be a rechargeable battery, such as lithium ion or lithium polymer battery, or may be a single-use battery such as alkaline or lithium battery. A battery meter 88 may provide the expected remaining power of the battery 86 to a user and/or to the microprocessor 48. In certain embodiments, the sensor 12 includes a charging interface that enables the sensor 12 to be coupled to the monitor 14 or a charger to charge the battery 86. In accordance with the present techniques, the sensor 12 may be configured to provide desired contact between the patient 46 and the detector 18, and/or the emitter 16. The sensor 12 may have varying levels of processing power, and may output data in various stages (e.g., during and/or after the administered CPR) to the monitor 14 or other device or network, either wirelessly (e.g., via wireless transceiver 92 of the sensor 12 and wireless transceiver 94 of the monitor 14) or via the cable 26. For example, in some embodiments, the data output to the monitor 14 may be analog signals, such as detected light signals (e.g., oximetry signals or regional saturation signals), or processed data.

As discussed above, the monitoring system 10 (e.g., sensor 12 and/or monitor 14) enable the analysis of physiological parameters (e.g., rSO₂ value) and/or signal characteristics to determine if the presently administered CPR needs to be adjusted, while also providing feedback with regard to one or more components of the CPR (e.g., chest compressions and/or artificial respiration). FIG. 6 generally illustrates a method 96 for utilizing the system 10 for this purpose. The method 96 begins with a responder (e.g., the person administering the CPR) applying the sensor 12 to the patient 46 (block 98). Monitoring of the physiological parameters (e.g., rSO₂) may then begin (block 100). As described above, the sensor 12 may detect activity by utilizing algorithms that detect a physiological signal or “sensor off” algorithms. The detected activity may trigger the sensor 12 and/or monitor 14 to begin monitoring. Alternatively, the input component 22 (e.g., actuation device) may be actuated on the sensor 12 and/or monitor 14 to mark the beginning of the monitoring. The responder may then begin administering CPR and the system 10 may begin analysis of the CPR (block 102). Alternatively, the responder may begin CPR before the application of the sensor, at which time the sensor 12 and monitor 14 immediately assesses the status of the ongoing CPR. Analysis of the CPR may be started by the responder providing an indication to the sensor 12 and/or monitor 14 (e.g., via actuation of the input component 22). The sensor 12 and/or monitor 14 may provide feedback (e.g., via display 20 and/or speaker 21) to the provider based on the rSO₂ value and analyzed signal characteristics as described above (block 104). The feedback may confirm the proper administration of the CPR, provide an analysis of the CPR, and/or provide adjustments for the administration of the CPR. The responder may then adjust the CPR based on the feedback (block 106). The monitoring, CPR, and CPR analysis may continue (block 108) until the termination of these activities (block 110). For example, the input component 22 (e.g., actuation device) may be actuated on the sensor 12 and/or monitor 14 to mark the ending of the monitoring, CPR, and CPR analysis. Following the ending of these activities, in embodiments where the sensor 12 functions as a standalone unit (i.e., conducts the CPR analysis and communicates feedback), data (e.g., patient's physiological data, CPR analysis data, etc.) may be transferred to the monitor 14 or a network (block 112). Alternatively, in embodiments where the monitor 14 performs the CPR analysis and communicates the feedback, the data may be transferred to the network or another system (e.g., electronic medical records).

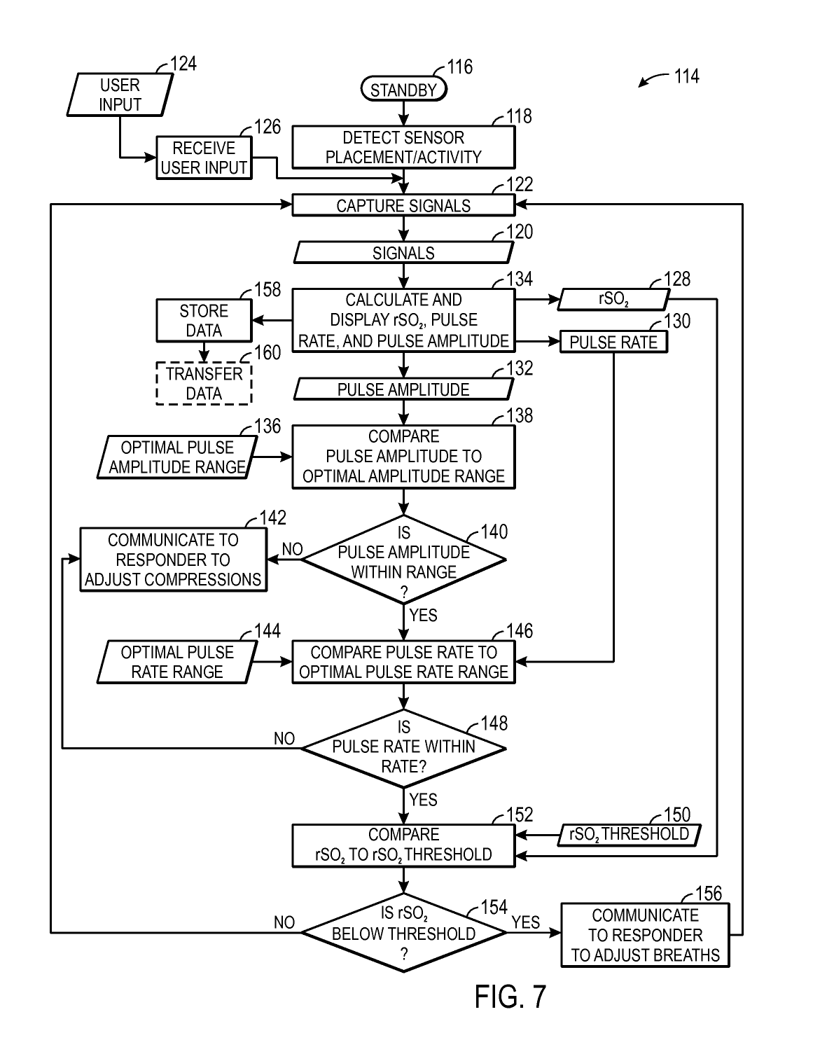

FIG. 7 provides a method 114 that illustrates the functionality of the monitoring system 10 in greater detail. The method 114 may begin with the sensor 12 applied to the patient functioning in a standby mode 116. In the standby mode 116, the sensor 12 may detect sensor placement or activity by utilizing algorithms that detect a physiological signal or “sensor off” algorithms (block 118). The detected activity may trigger the sensor 12 to begin capturing signals 120 (block 122). After application of the sensor 12, either prior to and/or after beginning to the capture the signals 120, the sensor 12 and/or monitor may receive user input 124 (block 126) (e.g., via the input component 22). For example, the placement of the sensor 12 and beginning the capture of signals may be indicated via user input 124. In addition, the beginning and ending of the administration of the CPR may be indicated via user input 124. Further, patient data (e.g., age, size, condition, etc.), threshold values, and/or ranges may be provided via user input 124. As described above, in certain embodiments, the sensor 12 and/or monitor 14 may select the strongest signal from among the signals 120 for subsequent analysis. The sensor 12 and/or monitor 14 may calculate and display from the signal 120 the rSO₂ value 128, pulse rate 130, and pulse amplitude 132 on the display 20 (block 134). The sensor 12 and/or monitor 14 may compare the pulse amplitude 132 to an optimal pulse amplitude range 136 (block 138). Based on this comparison, the sensor 12 and/or monitor 14 may determine whether the pulse amplitude 132 of the signal 120 is within the optimal pulse amplitude range 138 (block 140). If the pulse amplitude 132 is outside the optimal pulse amplitude range 138, the sensor 12 and/or monitor 14 may communicate (e.g., via display 20 and/or speaker 21) to the responder to adjust the chest compressions (e.g., location or amount of force applied) (block 142). If the pulse amplitude 132 is within the optimal pulse amplitude range 138, the sensor 12 and/or monitor 14 may compare the pulse rate 130 to an optimal pulse rate range 144 (e.g., 98 to 102 pulses/min, 96 to 104 pulses/min, 90 to 110 pulses/min, etc.) (block 146). In certain embodiments, the sensor 12 and/or monitor 14 may compare the pulse rate 130 to a threshold (e.g., minimum or maximum threshold for pulse rate). Based on this comparison, the sensor 12 and/or monitor 14 may determine whether the pulse rate 130 of the signal 120 is within the optimal pulse rate range 144 (block 148). If the pulse rate 130 is outside the optimal pulse rate range 144, the sensor 12 and/or monitor 14 may communicate (e.g., via display 20 and/or speaker 21) to the responder to adjust the chest compressions (e.g., location or amount of force applied) (block 142). If the pulse rate 130 is within the optimal pulse rate range 144, the sensor 12 and/or monitor 14 may compare the rSO₂ value 128 to an rSO₂ threshold 150 (block 152). Based on this comparison, the sensor 12 and/or monitor 14 may determine whether the rSO₂ value 128 is below the rSO₂ threshold 150. If the rSO₂ value 128 is below the rSO₂ threshold 150, the sensor 12 and/or monitor 14 may communicate (e.g., via display 20 and/or speaker 21) to the responder to adjust the breaths (e.g., amount and/or frequency) (block 156). If the rSO₂ value 128 is at or above the rSO₂ threshold 150, the sensor 12 continues capturing signals 120 and the sensor 12 and/or monitor 14 continue analyzing the signals 120. Although, the above method 114 discusses the analysis of pulse amplitude, pulse rate, and rSO₂ in a particular order, these steps may be performed in any order and/or simultaneously. The method 114 may also include storing the data acquired (e.g., rSO₂ value, pulse amplitude, pulse rate, analyses of the rSO₂ value and signals characteristics, patient characteristics, etc.) on the memory of the sensor 12 and/or monitor 14 (block 158). In certain embodiments, the stored data may be transferred (block 160) during and/or after the CPR and CPR analysis. For example, in embodiments where the sensor 12 functions as a standalone unit (i.e., conducts the CPR analysis and communicates feedback), data (e.g., patient's physiological data, CPR analysis data, etc.) may be transferred to the monitor 14 or a network. Alternatively, in embodiments where the monitor 14 performs the CPR analysis and communicates the feedback, the data may be transferred to the network or another system (e.g., electronic medical records).

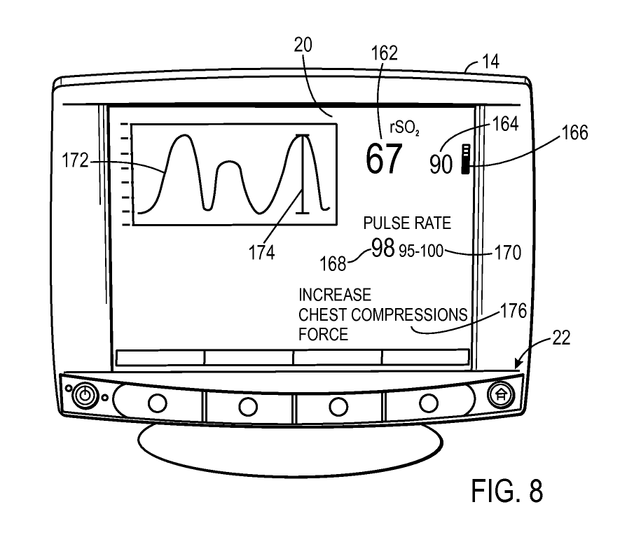

As described above, feedback with regard to the CPR may be communicated via display 20 of the monitor 14, for example, as illustrated in FIG. 8. It should be noted that a similar (although smaller) display 20 may be present on the sensor 12. In the arrangement shown in FIG. 8, the output from the sensor 12 is processed to provide an rSO₂ value or other such quantified value 162. In addition, a threshold rSO₂ value 164 and/or an indicator 166 illustrating the rSO₂ value relative to the rSO₂ threshold value on the display 20. Also, a pulse rate or frequency 168 along with a pulse rate range 170 may be shown on the display 20. In certain embodiments, a graphical representation 172 of the signal may be shown along with an indicator 174 for pulse amplitude. Suggested adjustments, directions, and other information 176 based on the analysis of the signal characteristics and the rSO₂ value may be shown on the screen. This information 176 may be shown in the form of text (as shown in FIG. 8), images, diagrams, symbols, or any other form to communicate the information 176 to the responder. The rSO₂ value 162, pulse rate 168, pulse amplitude indicator 174, and/or information 176 may be displayed or highlighted in different colors to (e.g., red, green, yellow, etc.) to indicate a state of that particular value or information. For example, an acceptable pulse rate, pulse amplitude, or non-urgent information (e.g., “Maintain Rate of Chest Compressions”) may be colored green, while unacceptable values or suggested adjustments may be colored red (e.g., “Increase Chest Compression Force”). As a result, information related to the CPR may be conveyed via the display 20, along with speaker 21 as described above, instantaneously to the responder to facilitate effective CPR administration.

In another embodiment, the monitor 14 could be a small handheld monitor including the display 20 and the speaker 21. However, the handheld monitor may display information in a manner understood by non-medically trained people. In other words, the handheld monitor may not display an rSO₂ value, pulse rate, graphs, or other items useful to medically trained people. Instead, the handheld monitor may display basic information (e.g., where to apply the sensor, how to conduct CPR, etc.) to the person providing the CPR as well as feedback to adjust the CPR (e.g., speed up compressions, give breath, etc.) via the display 20 and/or speaker 21. The feedback may include adjusting a pitch, tone, or other characteristic of the sounds emitted by the speaker 21 as described above. In addition, the handheld monitor may provide audible instructions via the speaker 21 and/or visible instructions via the display 20 as to how to apply the sensor 12, how to use the sensor 12 and/or monitor 14, and/or how to conduct the CPR. In some embodiments, the handheld monitor may include pictures or cards attached to illustrate how to apply the sensor 12 (e.g., where to place the sensor, how to use the sensor 12 and/or monitor 14, and/or how to conduct the CPR. The handheld monitor may include a sturdy outer case (e.g., rugged molded plastic shell cover) to protect the handheld monitor from fluid ingress. In certain embodiments, the handheld monitor may include default values (e.g., thresholds or ranges) set for the optimal pulse amplitude and/or pulse rate. In certain embodiments, the user may input patient characteristics (e.g., age, size, etc.) and the handheld monitor may be configured to select the pulse amplitude range, the pulse rate range, and/or the type of CPR to be administered (e.g., using heel of one hand for child, using two fingers for infant, using heels of both hands for adult, etc.) based on these patient characteristics. Alternatively, the handheld monitor may detect the type of sensor that is in use (e.g. adult, pediatric, infant) and select the type of CPR, target amplitudes, target rates, and/or target ranges based on the patient population for the selected sensor.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. An emergency resuscitation kit, comprising:
a regional oximetry sensor configured to be applied to a patient, comprising:
a light emitting element configured to emit light;
at least one light detector configured to receive the light and to generate a signal based on the received light; and
a monitor configured to receive the signal from the regional oximetry sensor, to analyze one or more characteristics of the signal to determine if cardiac pulmonary resuscitation (CPR) received by the patient needs to be adjusted, and to communicate to a person administering the CPR to adjust one or more components of the CPR based on the analysis.

2. The emergency resuscitation kit of claim 1, wherein analyzing one or more characteristics of the signal comprises determining a regional oxygen saturation value based on the received signal, and wherein the monitor is configured to communicate to the person administering the CPR to adjust the one or more components of the CPR based on the regional oxygen saturation value.

3. The emergency resuscitation kit of claim 3, wherein analyzing one or more characteristics of the signal further comprises comparing the regional oxygen saturation value to a threshold value to determine if the regional oxygen saturation value is lower than the threshold value, and wherein the one or more components of the CPR comprise breaths or other means of artificial respiration administered during the CPR to the patient.

4. The emergency resuscitation kit of claim 1, wherein the one or more characteristics of the signal comprise a pulse amplitude of the signal, and wherein analyzing the one or more characteristics comprises comparing the pulse amplitude of the signal to a pulse amplitude range to determine if the pulse amplitude is within the pulse amplitude range, and wherein the one or more components of the CPR comprise compressions administered during the CPR to the patient.

5. The emergency resuscitation kit of claim 1, wherein the one or more characteristics of the signal comprise a pulse rate of the signal, and wherein analyzing the one or more characteristics comprises comparing the pulse rate of the signal to a pulse rate range to determine if the pulse rate is within the pulse raterange, and wherein the one or more components of the CPR comprise chest compressions administered during the CPR to the patient.

6. The emergency resuscitation kit of claim 1, wherein the regional oximetry sensor comprises a plurality of light detectors configured to receive the light and to generate a respective plurality of signals based on the received light, and wherein the monitor is configured to receive the plurality of signals from the regional oximetry sensor and to select from among the plurality of signals the desired signals for analyzing the one or more characteristics.

7. The emergency resuscitation kit of claim 1, wherein the monitor is configured to automatically detect application of the regional oximetry sensor on the patient.

8. The emergency resuscitation kit of claim 1, wherein the one or more characteristics of the signal comprise a regional oxygen saturation value, a pulse amplitude, or a pulse rate, and wherein the analysis comprises comparing to a regional oxygen saturation threshold value, a pulse amplitude range, or a pulse rate range, respectively.

9. The emergency resuscitation kit of claim 8, wherein the monitor is configured to adjust one or more of the regional oxygen saturation threshold value, the pulse amplitude range, or the pulse rate range based on a user input related to patient characteristics.

10. The emergency resuscitation kit of claim 8, wherein the monitor is configured to adjust one or more of the regional oxygen saturation threshold value, the pulse amplitude range, or the pulse rate range based on predefined values associated with a patient population that corresponds to a sensor type of the regional oximetry sensor applied to the patient.

11. The emergency resuscitation kit of claim 1, wherein the monitor is further configured to provide audible feedback to the person administering the CPR indicating whether the CPR needs to be adjusted.

12. The emergency resuscitation kit of claim 11, wherein the one or more characteristics comprise a pulse rate of the patient, wherein the analysis comprises comparing the pulse rate to a pulse rate range, wherein the one or more components of the CPR comprise chest compressions, and wherein the audible feedback comprises a sound that indicates whether to adjust the chest compressions.

13. The emergency resuscitation kit of claim 12, wherein the sound changes in pitch to indicate to the person to increase or decrease a rate of administration of the chest compressions.

14. A regional oximetry sensor configured to be applied to a patient for use during administration of cardiac pulmonary resuscitation (CPR) to the patient, comprising:
a light emitting element configured to emit light;
a light detector configured to receive the light and generate a signal based on the received light; and
a processing device configured to receive the signal from the light detector, to analyze one or more characteristics of the signal to determine if the CPR administered to the patient needs to be adjusted, and to communicate to a person administering the CPR to adjust one or more components of the CPR based on the analysis.

15. The sensor of claim 14, wherein the processing device is configured to receive an input that the patient is receiving CPR, and wherein the regional oximetry sensor comprises an actuation device configured to receive the input.

16. The sensor of claim 15, wherein the actuation device comprises a knob, switch, key, keypad, button, or touchscreen.

17. The sensor of claim 14, wherein analyzing one or more characteristics of the signal comprises determining a regional oxygen saturation value based on the received signal, and wherein the processing device is configured to communicate to the person administering the CPR to adjust the one or more components of the CPR based on the regional oxygen saturation value.

18. The sensor of claim 17, wherein analyzing one or more characteristics of the signal further comprises comparing the regional oxygen saturation value to a threshold value to determine if the regional oxygen saturation value is lower than the threshold value, and wherein the one or more components of the CPR comprise breaths or other means of artificial respiration administered during the CPR to the patient.

19. The sensor of claim 14, wherein the one or more characteristics of the signal comprise a pulse amplitude of the signal, and wherein analyzing the one or more characteristics comprises comparing the pulse amplitude of the signal to a pulse amplitude range to determine if the pulse amplitude is within the pulse amplitude range, and wherein the one or more components of the CPR comprise chest compressions administered during the CPR to the patient.

20. The sensor of claim 14, wherein the one or more characteristics of the signal comprise a pulse rate of the signal, and analyzing the one or more characteristics comprises comparing the pulse rate of the signal to a pulse rate range to determine if the pulse rate is within the pulse rate range, and wherein the one or more components of the CPR comprise chest compressions administered during the CPR to the patient.

21. The sensor of claim 14, wherein the processing device is configured to automatically detect application of the regional oximetry sensor on the patient.

22. The sensor of claim 14, wherein the regional oximetry sensor comprises a display, and the processing device is configured to communicate to the person administering the CPR to adjust one or more components of the CPR via the display.

23. A regional oximetry monitor, comprising:
an interface for receiving a signal from a regional oximetry sensor applied to a patient receiving cardiac pulmonary resuscitation (CPR); and
a processing device configured analyze a pulse amplitude and a pulse rate of the signal to determine if the received CPR needs to be adjusted, and to communicate to a person administering the CPR to adjust the chest compressions administered during the CPR to the patient based on the pulse amplitude or pulse rate.

24. The monitor of claim 23, wherein the processing device is configured to determine a regional oxygen saturation value based on the received signal and to communicate to the person administering the CPR to adjust breaths administered during the CPR to the patient if the regional oxygen saturation value is lower than a threshold value.
